(19) United States
(12) Patent Application Publication
(10) Pub. No.: US 2021/0251528 A1
(43) Pub. Date: Aug. 19, 2021

(54) A glucose monitoring apparatus and method based on a fluorescence sensor

(71) Applicant: WITRACK, Gyeonggi-do (KR)

(72) Inventors: Sang Hoek KIM, Gyeonggi-do (KR); Yun Jung HEO, Gyeonggi-do (KR); Jong Heon LEE, Gyeonggi-do (KR)

(21) Appl. No.: 16/651,755

(22) PCT Filed: Aug. 29, 2019
(86) PCT No.: PCT/KR2019/011072

§ 371 Date: Mar. 27, 2020

(30) Foreign Application Priority Data
Nov. 8, 2018 (KR) 10-2018-0136456

(51) Int. Cl. A61B 5/145; A61B 5/1459

(52) U.S. Cl. CPC A61B 5/14532; A61B 5/0031; A61B 5/1459

(57) ABSTRACT

Glucose monitoring based on fluorescence sensor disclosed. A glucose monitoring apparatus according to one embodiment of the present invention may include a light emitter configured to emit an excitation light, a glucose sensor configured to absorb the excitation light and to emit a fluorescent light, a light receiver configured to receive the fluorescent light, and a circuit configured to modulate a scattered wave by using a series of pulses that are generated based on alight intensity of the fluorescent light.

### BACKGROUND

### Field

The present invention relates to a glucose monitoring apparatus and method based on a fluorescence sensor, more particularly, a technology of monitoring body glucose based on a fluorescent light emitted by a fluorescence glucose sensor.

### Related Arts

In starch metabolism, two hormones such as insulin and glucagon are secreted in a body.

In detail, insulin that is secreted from the pancreas. When the glucose level rises after having meals, the pancreas secretes insulin to prompt cells to take in glucose, and the liver synthesizes glycogen from glycose to store, thereby dropping the glucose level.

On the other hand, when the glucose level drops as time passes, the pancreas reduces insulin secretion, and in return, secretes glucagon for the liver to convert glycogen into glucose to flow into blood, thereby raising the glucose level.

The glucose concentration in blood has a close relationship with diseases related to this metabolism such as diabetes, and hyperglycemia and hypoglycemia due to diabetes. In prevention, diagnosis, and treatment of disease, measuring blood sugar level is the most important means.

Especially, since hypoglycemia may cause shock to a diabetic patient to lead to death, it is very important to continuously measure glucose level of the diabetic patient. Interests in development of implantable sensor are growing for continuous monitoring of glucose level.

But, conventional implantable sensors are using digital communication, which consumes more power than analog communication.

Specifically, the conventional implantable sensors which uses digital communication require several circuits such as ADC(Analog-Digital Converter), Memory, CPU(Central processing unit), and Bluetooth module, which consume much power and cause to increase the size of sensor.

### SUMMARY

The present invention is intended to provide a glucose monitoring apparatus and method, which can minimize power consumption and size of apparatus by using analog communication such as scattered wave modulation, rather than digital communication.

In addition, the present invention is intended to provide a glucose monitoring apparatus and method, which can block noises occurring due to excitation light in glucose measurement data in advance by using a filter.

Also, the present invention is intended to provide a glucose monitoring apparatus and method, of which size can be further reduced by implementing a modulator with a single transistor.

A glucose monitoring apparatus according to one embodiment of the present invention may include a light emitter configured to emit an excitation light, a glucose sensor configured to absorb the excitation light and to emit a fluorescent light, a light receiver configured to receive the fluorescent light, and a circuit configured to modulate a scattered wave by using a series of pulses that are generated based on a intensity of the fluorescent light.

According to one aspect, the glucose monitoring apparatus may further include an antenna configured to receive an alternating signal from an outside and provide it to the circuit.

According to one aspect, the glucose monitoring apparatus may be implanted to a subcutaneous tissue at a depth between 1 mm and 2 mm below an epidermis of a patient.

According to one aspect, the glucose sensor may be configured to emit the fluorescent light having the light intensity that varies in proportion with a glucose concentration in a patient.

According to one aspect, the light receiver may include a filter configured to block the excitation light.

According to one aspect, the circuit may include a pulse generator configured to generate the series of pulses having a pulse period corresponding to the light intensity of the fluorescent light, and a modulator configured to modulate the scattered wave that is a portion of an incoming wave from the outside reflected by the antenna.

According to one aspect, the modulator includes at least one transistor configured to receive the series of pulses generated by the pulse generator as gate input and to modulate an amplitude of the scattered wave.

According to one aspect, the circuit may further include a rectifier configured to rectify the alternating signal received from the outside, and a regulator configured to regulate a voltage of the rectified signal and to provide a regulated signal to the light emitter.

A glucose monitoring method according to one embodiment of the present invention may include emitting an excitation light in a light emitter, emitting a fluorescent light when receiving the excitation light in a glucose sensor, receiving the fluorescent light in a light receiver, and modulating a scattered wave by using a series of pulses generated based on an intensity of the fluorescent light in a circuit.

According to one aspect, the emitting an excitation light in the light emitter may include providing an alternating signal received by an antenna from an outside to a rectifier of the circuit, rectifying the alternating signal received from the outside in the rectifier, regulating a voltage of the rectified signal and providing the regulated signal to the light emitter in a regulator of the circuit, and emitting the excitation light based on the regulated signal in the light emitter.

According to one aspect, the emitting a fluorescent light when receiving the excitation light in the glucose sensor may be the emitting the fluorescent light having light intensity that varies in proportion with a glucose concentration in the patient.

According to one aspect, wherein the modulating the scattered wave by using the series of pulses generated based on the light intensity of the fluorescent light in the circuit may include generating the series of pulses having a pulse period that a pulse generator of the circuit generates corresponding to the light intensity of the received fluorescent ray, and modulating the scattered wave that is the portion of an incoming wave from an outside reflected by an antenna in a modulator.

According to one embodiment, the power consumption and the size of apparatus can be minimized by using analog communication such as scattered wave modulation, rather than digital communication.

In addition, according to one embodiment, noise occurring due to reception of excitation light in glucose measurement data can be blocked in advance by use of filter.

In addition, according to one embodiment, the size of apparatus can be further reduced by implementing the modulator with a single transistor.

In addition, according to one embodiment, the size of apparatus can be further reduced by implementing the entire circuit system in integrated chip.

### BRIEF DESCRIPTION OF DRAWINGS

FIG. 1 schematically illustrates a glucose monitoring apparatus according to one embodiment;

FIG. 2 is a schematic block diagram of circuit in the glucose monitoring apparatus according to one embodiment;

FIG. 3 illustrates pulses generated by a pulse generator in the glucose monitoring apparatus according to one embodiment;

FIGS. 4a and 4b schematically illustrate a modulator in the glucose monitoring apparatus according to one embodiment; and

FIG. 5 is a flowchart of glucose monitoring method according to one embodiment.

### DETAILED DESCRIPTION

Embodiments of the present disclosure are described with reference to the accompanying drawings.

This disclosure, however, should not be construed as limited to the exemplary embodiments and terms used in the exemplary embodiments, and should be understood as including various modifications, equivalents, and substituents of the exemplary embodiments.

In the description of embodiments of the present disclosure, certain detailed explanations of related known functions or constructions are omitted when it is deemed that they may unnecessarily obscure the essence of the disclosure.

In addition, the terms used in the specification are defined in consideration of functions used in the present disclosure, and can be changed according to the intent or conventionally used methods of clients, operators, and users. Accordingly, definitions of the terms should be in the drawings, like reference numerals in the drawings denote like elements.

The same reference symbols are used throughout the drawings to refer to the same or like parts.

As used herein, the singular forms “a,” “an” and “the” are intended to include the plural forms as well, unless context clearly indicates otherwise.

Expressions such as “A or B” and “at least one of A and/or B” should be understood to include all possible combinations of listed items.

Expressions such as “a first,” “the first,” “a second” and “the second” may qualify corresponding components irrespective of order or importance and may be only used to distinguish one component from another component without being limited to the corresponding components.

In the case in which a (e.g., first) component is referred as “(functionally or communicatively) connected” or “attached” to another (e.g., second) component, the first component may be directly connected to the second component or may be connected to the second component via another component (e.g., third component).

In the specification, the expression “configured to (or set to)” may be used interchangeably, for example, with expressions, such as “suitable for,” “having ability to,” “modified to,” “manufactured to,” “enabling to,” or “designed to,” in the case of hardware or software depending upon situations.

In any situation, the expression “an apparatus configured to” may refer to an apparatus configured to operate “with another apparatus or component.”

For examples, the expression “a processor configured(or set) to execute A, B, and C” may refer to a specific processor performing a corresponding operation (e.g., embedded processor), or a general-purpose processor (e.g., CPU or application processor) executing one or more software programs stored in a memory device to perform corresponding operations.

In addition, the expression “or” means “inclusive or” rather than “exclusive or”.

That is, unless otherwise mentioned or clearly inferred from context, the expression “x uses a or b” means any one of natural inclusive permutations.

In the above-described detailed embodiments of the disclosure, a component included in the disclosure is expressed in the singular or the plural according to a presented detailed embodiment.

However, the singular form or plural form is selected for convenience of description suitable for the presented situation, and various embodiments of the disclosure are not limited to a single element or multiple elements thereof. Further, either multiple elements expressed in the description may be configured into a single element or a single element in the description may be configured into multiple elements.

Although the embodiment has been described in the detailed description of the disclosure, the disclosure may be modified in various forms without departing from the scope of the disclosure.

Therefore, the scope of the disclosure should not be defined as being limited to the embodiments, but should be defined by the appended claims and equivalents thereof.

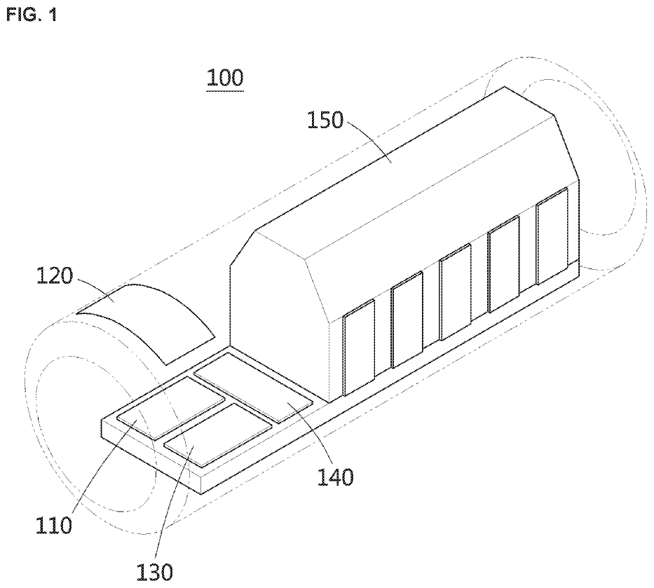

FIG. 1 schematically illustrates a glucose monitoring apparatus according to one embodiment

Referring to FIG. 1, the glucose monitoring apparatus 100 according to one embodiment uses analog communication such as a scattered wave modulation instead of digital communication, thereby minimizing power needed for the apparatus and size of the apparatus.

For this, the glucose monitoring apparatus 100 may include a light emitter 110, a glucose sensing sensor 120, a light receiver 130, and a circuit 140.

According to one aspect of embodiment, the glucose monitoring apparatus 100 may further include an antenna 150 that is coupled to the circuit 140 to provide an alternating signal received from the outside to the circuit 140.

In addition, the glucose monitoring apparatus 100 may be implanted to a subcutaneous tissue at a depth between 1 mm and 2 mm below an epidermis of the patient.

Namely, the glucose monitoring apparatus 100 according to one embodiment can be implanted to the patient's body, and be configured to receive power and transmit monitoring result wirelessly, thereby monitoring continuously glucose level without blood collection.

In detail, the light emitter 110 is configured to emit an excitation light. For example, the light emitter 110 may include more than one light emitting diode (LED).

The glucose sensor 120 may be configured to absorb the excitation light to emit a fluorescent light.

According to one aspect of embodiment, the glucose sensor 120 may be configured to emit a fluorescent light having light intensity that varies in proportion with a glucose concentration in the patient.

In detail, the glucose sensor 120 may include a fluorescent material. The fluorescent material in the glucose sensor 120 may emit the fluorescent light in response to the excitation light from the light emitter 110.

As the glucose concentration in the patient's body is high, an intensity of the fluorescent light emitted by glucose sensor 120 may become stronger.

The light receiver 130 according to one embodiment may be configured to receive the fluorescent light. For example, the light receiver 130 may include more than one photodiode.

According to one aspect, the light receiver 130 may include a filter configured to block the excitation light.

In detail, as a wavelength of the excitation light is shorter than that of the fluorescent light, the excitation light into the light receiver 130 can be blocked in advance.

The light receiver 130 may be configured to receive the fluorescent light from the glucose sensor 120, and eliminate noise that occur in glucose measurement data due to reception of the excitation light in advance with the filter configured to block the excitation light.

For example, the light receiver 130 may include a filter capable of passing light in a certain wavelength range in which the fluorescent light belongs to. On the contrary, the light receiver 130 may include a filter capable of blocking light in a certain wavelength range in which the excitation light belongs to.

The circuit 140 according to one embodiment may be configured to modulate a scattered wave by using pulses that are generated based on the light intensity of the fluorescent light received by the light receiver 130.

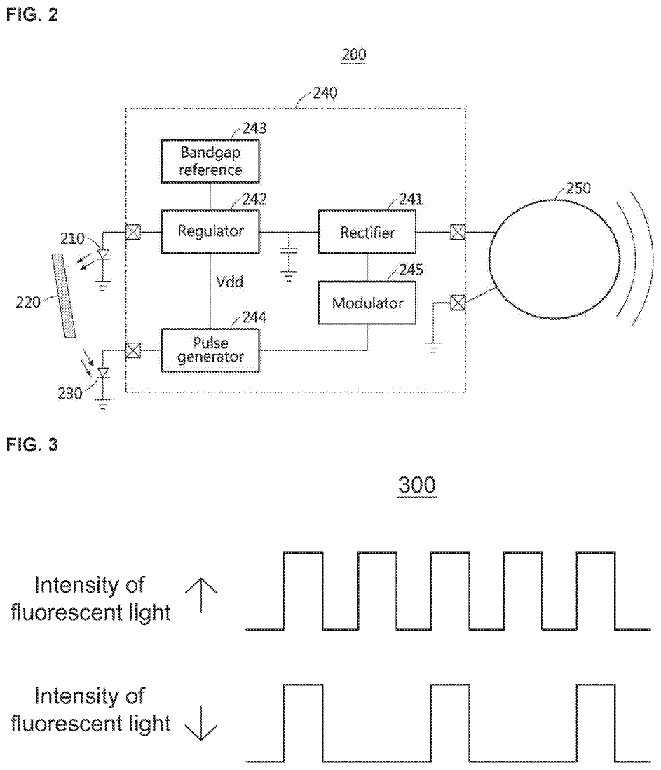

Configuration of the circuit 140 according to one embodiment will be described in detail with reference to FIG. 2.

FIG. 2 is a schematic block diagram of circuit in the glucose monitoring apparatus according to one embodiment.

Referring to FIG. 2, a light emitter 210 of a glucose monitoring apparatus 200 according to one embodiment may be configured to emit the excitation ray, and a glucose sensor 220 may be configured to absorb the excitation light to emit a fluorescent light.

A light receiver 230 may be configured to receive the fluorescent light and to provide data corresponding to the received fluorescent light to a circuit 240.

In addition, since the fluorescent light received by the light receiver 230 has a very low body permeability, it is difficult to secure a reliability in monitoring result when data corresponding to the fluorescent light is conveyed intactly to the outside.

Accordingly, the glucose monitoring apparatus 200 modulates, by the circuit 240, data corresponding to the fluorescent light to convey to the outside with no loss of data so that the reliability in monitoring result can be secured.

According to one aspect, the circuit 240 may include a pulse generator 244 configured to generate a series of pulses having a pulse period corresponding to the light intensity of the fluorescent light.

In detail, the pulse generator 244 may be configured to output pulses with different pulse periods corresponding to a voltage that varies according to the light intensity of the fluorescent light received by the light receiver 230.

For example, when the voltage rises as the light intensity of the fluorescent light goes high, the pulse generator 244 may generate pulses with a short pulse period corresponding to relatively high voltage.

On the contrary, when the voltage drops as the light intensity of the fluorescent light goes low, the pulse generator 244 may generate pulses with a long pulse period corresponding to relatively low voltage.

An example that the pulse generator 244 generates pulses according to one embodiment will be described in detail with reference to FIG. 3.

According to one aspect, the circuit 240 may include a modulator 245 configured to, with pulses generated by the pulse generator 244, modulate a scattered wave that is a portion of an incoming wave from the outside reflected by an antenna 250.

By the modulator 245, a portion of incoming wave that is received from an external device and then reflected by the antenna 250 may be amplitude modulated.

For example, a signal from the outside received by the antenna 250 may be the alternating signal that is received from the outside by the antenna 250 and then provided to a rectifier 241. The signal from the outside received by the antenna 250 may be another signal distinguishably received from the AC signal that is provided to the rectifier 241.

Amplitude-modulated scattered wave that is propagated to the outside can be detected by the external device. The external device can monitor the glucose concentration in the patient's body by demodulating the amplitude-modulated scattered wave.

Namely, the external device may demodulate the amplitude-modulated scattered wave, generate a voltage value from the demodulated scattered wave, and generate the glucose concentration corresponding to the voltage value.

In the present invention, it becomes possible to minimize the amount of power needed to operate the glucose monitoring apparatus 200 and the size of the same by using, instead of digital communication, the scattered wave modulation that is a kind of analog communication.

In detail, if the glucose monitoring apparatus 200 had a circuit that can generate a wireless signal being modulated according to pulses from the pulse generator 244 and actively propagate it to the outside, the amount of power needed will increase and the size of apparatus will increase as well.

Instead of generating a modulated signal and outputting the modulated signal by itself, the glucose monitoring apparatus 200 according to one embodiment has the circuit 240 configured to modulate the amplitude of scattered wave that is reflected by the antenna 250 to provide measured glucose data to the outside, thereby minimizing the amount of power and the size.

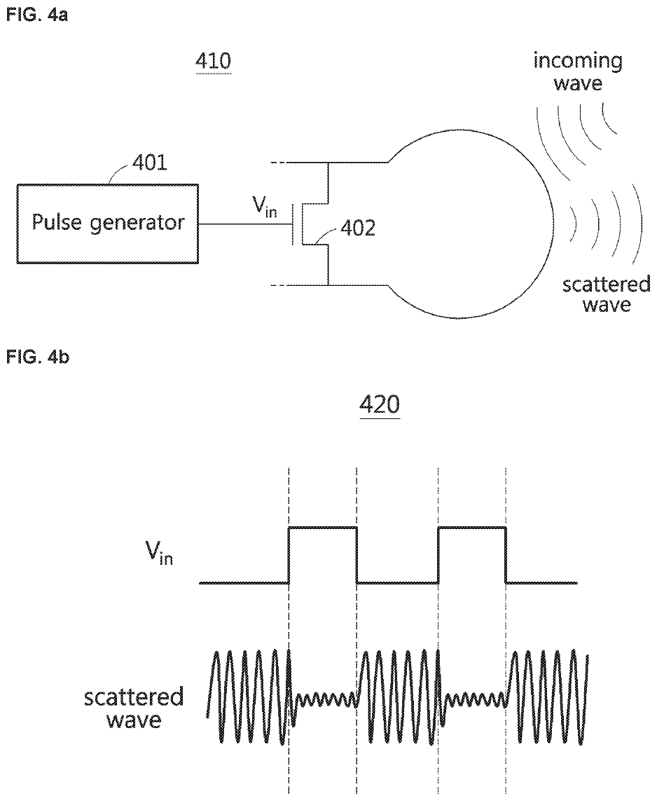

Operation of the modulator 245 will be described in detail with reference to FIGS. 4a and 4b.

According to one aspect, the circuit 240 may further include a rectifier 241 configured to rectify the alternating signal received from the outside, and a regulator 242 configured to regulate the rectified signal and to provide the regulated signal to the light emitter 210.

In addition, the circuit 240 may further include a bandgap reference voltage generator 243 configured to generate and provide a reference voltage to the voltage regulator 242.

In detail, the rectifier 241 may receive the alternating signal received through the antenna 250 from the outside, and rectify the received alternating signal to provide to the regulator 242.

The alternating signal received from the outside is a wirelessly-transferred power.

The regulator 242 may be configured to regulate voltage of the rectified signal, and provide the regulated signal Vdd to the light emitter 210 and/or the pulse generator 244.

The light emitter 210 may operate with the regulated signal Vdd received from the regulator 244.

FIG. 3 illustrates pulses generated by a pulse generator in the glucose monitoring apparatus according to one embodiment.

Referring to FIG. 3, as indicated by reference numeral 300, the pulse generator in the glucose monitoring apparatus according to one embodiment may be configured to generate a series of pulses with a relatively shorter pulse period as the light intensity of fluorescent light becomes strong.

On the contrary, the pulse generator in the glucose monitoring apparatus according to one embodiment may be configured to generate a series of pulses with a relatively longer pulse period as the light intensity of fluorescent light becomes weak.

FIGS. 4a and 4b schematically illustrate a modulator in the glucose monitoring apparatus according to one embodiment.

Referring to FIGS. 4a and 4b, as indicated by reference numeral 410, the modulator may include a transistor 402 configured to receive pulses Vin generated by the pulse generator 401 as gate input and to modulate the amplitude of the scattered wave.

The modulator may be configured to receive pulses Vin as input and modulate the amplitude of the scattered wave that is a portion of an incoming wave from the outside reflected by an antenna 250.

In detail, the modulator, as indicated by reference numeral 420, may be configured to modulate the amplitude of the scattered wave by turning the transistor 402 on and off in response to pulses Vin that are provided to a gate of the transistor 402.

A waveform of the scattered wave modulated by N-type transistor and a waveform of the scattered wave modulated by P-type transistor may be in opposite shape when turning the transistor 402 on and off.

Namely, the present invention implements the modulator with a single transistor to further minimize the size of the glucose monitoring apparatus.

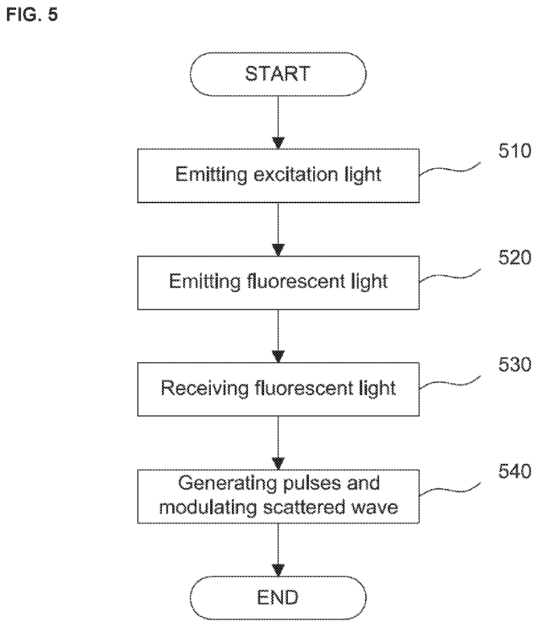

FIG. 5 is a flowchart of glucose monitoring method according to one embodiment.

Hereinafter the method described with reference to FIG. 5 relates to a monitoring method using the glucose monitoring apparatus according to one embodiment, so same description that is already described above with reference to FIGS. 1 through 4b will be omitted.

Referring to FIG. 5, at step 510, the glucose monitoring method according to one embodiment may emit the excitation light in the light emitter.

According to one aspect, at step 510, the glucose monitoring method according to one embodiment may receive the alternating signal from the outside and provide to the rectifier the antenna, and rectify the alternating signal received from the outside in the rectifier.

In addition, at step 510, the glucose monitoring method according to one embodiment may regulate the voltage of the rectified signal and to provide the regulated signal to the light emitter in the regulator of the circuit, and emit the excitation light based on the regulated signal in the light emitter.

At step 520, the glucose monitoring method according to one embodiment may absorb the excitation light and to emit the fluorescent light in the glucose sensor.

According to one aspect, at step 520, the glucose monitoring method according to one embodiment may emit a fluorescent light having light intensity that varies in proportion with a glucose concentration in the patient in the glucose sensor.

At step 530, the glucose monitoring method according to one embodiment may receive the fluorescent light in the light receiver.

At step 540, the glucose monitoring method according to one embodiment may modulate the scattered wave by using pulses that the circuit generates based on the light intensity of the received fluorescent light.

According to one aspect, at step 540, the glucose monitoring method according to one embodiment may generate pulses with the pulse period corresponding to the light intensity of the received fluorescent light in the pulse generator, and modulate the scattered wave that is the portion of the incoming wave from the outside reflected by the antenna in the modulator.

In the present invention, it becomes possible to minimize the amount of power needed to operate the glucose monitoring apparatus 200 and the size of the same by using, instead of digital communication, the scattered wave modulation that is a kind of analog communication.

In addition, the present invention may block noises occurring due to excitation light in glucose measurement data in advance by using a filter.

Also, the present invention can be further reduce the size of the glucose monitoring apparatus by implementing a modulator with a single transistor.

The aforementioned device may be realized by a hardware component, a software component, and/or a combination of hardware and software components. For example, the device and components described in the embodiments may be realized using one or more general-purpose computers or special-purpose computers such as, for example, a processor, a controller, an arithmetic logic unit (ALU), a digital signal processor, a microcomputer, a field programmable array (FPA), a programmable logic unit (PLU), a microprocessor, or other devices implementing instructions and responding thereto. The processor may execute one or software applications that run on an operating system (OS). In addition, the processor may approach data, store, manipulate, and process the data, and generate new data by responding to running of software. Although one processor has been used to aid in understanding, those skilled in the art can understand that the processor may include a plurality of processing elements and/or a plurality of processing element types. For example, the processor may include a plurality of processors or a combination of one processor and controller. Further, another processing configuration, such as a parallel processor, may be applied.

Software may include a computer program, code, instructions, or a combination of one or more of the foregoing, and may configure a processing device to operate as desired or independently or collectively a command to a processing device. Software and/or data may be permanently or temporarily embodied in the form of any type of machines, components, physical devices, virtual equipment, computer storage media or devices, or a signal wave to be transmitted, so as to be interpreted by a processing device or to provide a command or date to a processing device.

Software may be distributed over a networked computer system, and stored or executed in a distributed manner. Software and data may be stored on one or more computer readable media.

Embodiments of the present disclosure can include a computer readable medium including program commands for executing operations implemented through various computers. The computer readable medium can store program commands, data files, data structures or combinations thereof. The program commands recorded in the medium may be specially designed and configured for the present disclosure or be known to those skilled in the field of computer software. Examples of a computer readable recording medium include magnetic media such as hard disks, floppy disks and magnetic tapes, optical media such as CD-ROMs and DVDs, magneto-optical media such as floptical disks, or hardware devices such as ROMs, RAMs and flash memories, which are specially configured to store and execute program commands. Examples of the program commands include a machine language code created by a compiler and a high-level language code executable by a computer using an interpreter and the like. The hardware devices may be configured to operate as one or more software modules to perform operations in the embodiments, and vice versa.

What is claimed is:

1. A glucose monitoring apparatus, comprising:
a light emitter configured to emit an excitation light;
a glucose sensor configured to absorb the excitation light and to emit a fluorescent light;
a light receiver configured to receive the fluorescent light; and
a circuit configured to modulate a scattered wave by using a series of pulses that are generated based on a light intensity of the fluorescent light.

2. The glucose monitoring apparatus of claim 1 further comprising an antenna configured to receive an alternating signal from an outside and provide to the circuit.

3. The glucose monitoring apparatus of claim 1, wherein the apparatus is implanted to a subcutaneous tissue at a depth between 1 mm and 2 mm below an epidermis of a patient.

4. The glucose monitoring apparatus of claim 1, wherein the glucose sensor is configured to emit the fluorescent light having the light intensity that varies in proportion with a glucose concentration in a patient.

5. The glucose monitoring apparatus of claim 1, wherein the light receiver includes a filter configured to block the excitation light.

6. The glucose monitoring apparatus of claim 2, wherein the circuit comprises:
a pulse generator configured to generate the series of pulses having a pulse period corresponding to the light intensity of the fluorescent light; and
a modulator configured to modulate the scattered wave that is a portion of an incoming wave from the outside reflected by the antenna.

7. The glucose monitoring apparatus of claim 6, wherein the modulator includes at least one transistor configured to receive the series of pulses generated by the pulse generator as gate input and to modulate an amplitude of the scattered wave.

8. The glucose monitoring apparatus of claim 2, wherein the circuit further comprises:
a rectifier configured to rectify the alternating signal received from the outside; and
a regulator configured to regulate a voltage of the rectified signal and to provide a regulated signal to the light emitter.

9. A glucose monitoring method, comprising:
emitting an excitation light in a light emitter;
emitting a fluorescent light when receiving the excitation light in a glucose sensor;
receiving the fluorescent light in a light receiver; and
modulating a scattered wave by using a series of pulses generated based on a light intensity of the fluorescent light in a circuit.

10. The glucose monitoring method of claim 9, wherein the emitting an excitation light in the light emitter comprises:
providing an alternating signal received by an antenna from an outside to a rectifier of the circuit;
rectifying the alternating signal received from the outside in the rectifier;
regulating a voltage of the rectified signal and providing the regulated signal to the light emitter in a regulator of the circuit; and
emitting the excitation light based on the regulated signal in the light emitter.

11. The glucose monitoring method of claim 9, wherein the emitting a fluorescent light when receiving the excitation light in the glucose sensor is the emitting the fluorescent light having light intensity that varies in proportion with a glucose concentration in the patient.

12. The glucose monitoring method of claim 9, wherein the modulating the scattered wave by using the series of pulses generated based on the light intensity of the fluorescent light in the circuit comprises:
generating the series of pulses having a pulse period that a pulse generator of the circuit generates corresponding to the light intensity of the received fluorescent light; and
modulating the scattered wave that is the portion of an incoming wave from an outside reflected by an antenna in a modulator.
